(19) United States
(12) Patent Application Publication
(10) Pub. No.: US 2006/0008908 A1
(43) Pub. Date: Jan. 12, 2006

(54) Method and composition for longevity assurance

(76) Inventor: Brian C. Giles, Montecito, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/178,050

(22) Filed: Jul. 8, 2005

Related U.S. Application Data
(60) Provisional application No. 60/586,467, filed on Jul. 8, 2004.

(51) Int. Cl. C12N 15/63; C12N 5/08

(52) U.S. Cl. 435/455; 435/366

(57) ABSTRACT

This present invention provides compositions and methods for Longevity Assurance and treatment of disorders associated with age-related diseases. The compositions and methods further address age-related or incorrect or abnormal regulation of cellular homeostasis by controlling mechanisms of systemic, intracellular and extracellular ionic physiology through the administration of alkaline salts. The therapy described corrects molecular and ionic pathology, promoting Longevity Assurance with disease resistance.

### CROSS REFERENCE TO RELATED APPLICATIONS

This application claims priority to U.S. Provisional Patent Application Ser. No. 60/586,467 filed on Jul. 8, 2004, the entire disclosure of which is incorporated by reference.

### STATEMENT REGARDING FEDERALLY SPONSORED RESEARCH OR DEVELOPMENT

Not applicable.

### REFERENCE TO A MICROFICHE APPENDIX

Not applicable.

### TECHNICAL FIELD

The present invention relates to the fields of pharmacology, regenerative medicine and nutricology. It is intended for treatment of metabolic disorders associated with aberrant regulation of cellular homeostatis, for aging intervention and expansion of mortality, for treatment of age-related diseases, and for providing nutritional supplementation.

### BACKGROUND INFORMATION AND DISCUSSION OF RELATED ART

The prior art has primarily consisted of treating symptoms associated with degenerative diseases and does not address the electro-physiological origins of aging and disease. Known drugs often adversely affect ionic functions and compromise and the essential systemic pH, cellular pHe, cellular pHi and the mitochondria pHe and pHi function. With age progression, systemic pH, pHe and pHi are progressively more sub-optimal. The pHe acidosis (extra cellular acidosis) is a condition commonly associated with a wide variety of physiological and pathological situations. This may be largely due to the fact that pH regulates gene expression.

This (reduced) suboptimal pH lowers the critical enzymatic activities of normal, healthy viable cells and their ability to assimilate and metabolize nutrients. It further compromises cellular repair and replication, reduces cellular metabolic processes, promotes shape changes and affects function that substantially contributes to cellular life span.

For example, high blood pressure can be treated with drugs that disrupt normal homeostatic mechanisms governing fluid retention. This strategy, however, puts the patient at risk from the side effects of regulation loss. A superior, alternative strategy would involve the correction of underlying ionic imbalance, so that excess sodium retention is detected and corrected. Correct homeostasis is restored instead of selectively destroyed.

It would be advantageous to have compositions that satisfy a need in the art by providing alkaline ionic compositions that are useful in the prevention and treatment of disorders associated with aberrant regulation of cellular homeostasis and disorders associated with extension of senescence (aging) cycle and disease resistance.

The present invention is separate from and superior to the above-referenced art, eliminating the short and long term harmful side effects prevalent with existing therapies.

### SUMMARY OF THE INVENTION

Traditional therapies do not take into account the underlying electro-physiological and electro-biological consequences responsible for the aging process and cell dysregulation, nor do they provide for good health or Longevity Assurance; generally, they only provide relief from the symptoms of ill health. It is a far better strategy to provide pH-restoring ionic compositions that support the optimum homeostatic regulation of systemic pH, cellular pHe and cellular pHi (including mitochondrial pHe and pHi) and other aspects of cellular electrophysiology.

The pH-restoring ionic compositions of the present invention optimize the electro-physiological environment for cells in general, including healthy cells. This optimization ensures that cellular biochemistry stays fully functional, and tissue repair, metabolism and immune function are maintained during the aging process.

Aging and age-related diseases are induced by the dysregulation of viscosity, resistivity, and the chronic loss of pH homeostatis. The aging process is the accumulation of acid wastes (H+) in the pHe (exo-cellular) and pHi (intra-cellular) environment. The aging process can be slowed, stabilized and/or reversed by removing stored, acidic wastes and the restoration of the proton path. Thus, an alkalinizing composition and method is highly effective at restoring the aging cell to its optimum or near optimum function, i.e., restoring and reversing the aging process.

Genetic damage occurs in a sub-optimum pH, and free radicals propagate in a sub-optimum pH. Even a minor elevation in pHi toward an optimum range extends cellular lifespan and replication cycle and increases its function. The pH values control and maintain the DNA and messenger RNA structure and function for the generation of new repair proteins.

Healthy cells have a greater surface area, structural conformation, energy, elasticity and tensegrity. The life cycle of healthy aerobic human cells generally involves optimally 50-100 cell divisions before terminal differentiation occurs. In senescence (aging) and under a sub-optimal pH, cells lose structural energy, ellipse, enlarge and metabolize protein inefficiently; they are no longer able to divide. An optimum pHe ranges between 7.31 to 7.44. pH controls the “counting” gene that determines the number of times (cycles) that human cells are able to divide before terminal differentiation occurs.

The pH determines the duration of the cell replication cycle. Telomeres lose segments at chromosomal ends during each cell division, due to a loss of template energy that occurs in a sub-optimum pHi, i.e., aging or cell and tissue necrosis. Telomerases, the enzymes associated with telomeres, serve as formatting templates to rejuvenate older or damaged cells and to maintain a stable length of telomerase on the ends of the telomeres. By obtaining an optimal, cellular electro-physiological environment (pHe ranging from 7.31 to 7.44, preferably 7.40), the reproduction environment of the longevity enzyme telomerase is maintained and the replication cycles are extended.

By administering a composition of the present invention, systemic pH, pHe and pHi are restored to an optimal or near optimal electro-physiological environment. This supports the infrastructure of enzymes that repair proteins, reducing recovery time and reinforcing the structural integrity of all proteins. It further promotes and facilitates the release of energy from the energy (currency) molecule ATP in cell mitochondria (powerhouses) and improves the transport of nutrients and oxygen to cells throughout the body.

Optimizing the pHe to a range between 7.31 to 7.44 makes oxygen available for complete glucose metabolism. Glucose is the primary cell food. Alkalinizing biological fluids to obtain and maintain a physiologically optimum pHe of 7.31 to 7.44 makes oxygen available for complete glucose metabolism and promotes the replication of telomerase.

Proton channels exist in a wide variety of membrane proteins, where they transport protons rapidly and efficiently. Usually the proton pathway is formed with water molecules present in the protein and is regulated by titratable groups on critical amino acid residues in the pathway. The proton channels conduct protons by a hydrogen-bonded chain mechanism in which the proton hops from one water or titratable group to the next. Voltage-gated proton channels represent a specific subset of proton channels that have voltage- and time-dependent gating like other ion channels. They differ from most ion channels, however, in their extremely high selectivity, minute conductance, strong temperature and deuterium isotope effects on conductance and gating kinetics, and insensitivity to block by steric occlusion. The gating of the H+ (ion) channels is closely regulated by pHe and pHi and voltage, ensuring that they open only when the electrochemical gradient is outward. Thus, they function to extrude acid from inside the cells.

Compositions of the present invention include the ions of cesium and/or rubidium, the two most alkaline ions, which provide the alkaline ions to restore the systemic pH, pHe, and pHi necessary for Longevity Assurance. The composition's cesium and rubidium salts dissociate to the ionic form of Cesium and Rubidium. Cesium and rubidium ions reduce or eliminate the H+ in cells and tissues. There is variability in the ionization capability of the various salts of cesium and rubidium with some ionizing more readily than others. Any combination of cesium and/or rubidium salts that disassociate and ionize may be employed, including, but not limited to the acids: Arginate, Ascorbate, Caprylate, Cysteinate, Citrate, Fumarate, Methionine, Glutamate, Gluconate, Glycinate, Aspartate, Lysinate, Succinate, Carbonate, Lactate, Malate, Tartrate, Chloride, Sulfate, Phosphate, Nitrate, Bromide, Iodide.

Compositions of the present invention eliminate the free radical propagation environment that occurs in a sub-optimum pH/pHe/and pHi environment. Such compositions electro-chemically/electro-physiologically neutralize a broad spectrum of acidic substances associated with cellular morbidity, i.e., the primary source of the aging process, obtaining efficient hydration, therefore improving metabolic function transport and slowing the aging process.

There are many drugs and therapies aimed at correcting specific disease symptoms that result from failure to maintain the ideal physiological conditions. Preventative protection of the body from failure of homeostasis has generally been relegated to the realm of nutritional and lifestyle choices. The knowledge and understanding of pH regulation and ionic physiology enables the provision of compositions and methods that make gene protection and stabilization, e.g., the accumulation of genetic damage within cells, accessible and convenient. This results in profound benefits to human health and mortality expansion.

The compositions and methods of the present invention shift (increase) the negative hydrogen (ions) in the body fluids. The electrons move the fluids to an ORP and restore the sub-optimum pHi to more optimum levels that potentiate the optimum function of immune function and response and apoptosis. The balance between aging and age-related diseases is shifted to promote repair and regeneration by promoting optimum electro-physical cellular function. Secondarily, by stabilizing or reducing systemic and localized acidosis, i.e., inflammation, and increasing oxygenation in the electro-biological environment, the hostile effects caused by acidosis (sub-optimum pH) are sub-optimum or eliminated and optimum pH values are restored. This reverses molecular and ionic pathology and restores cellular electrochemical equilibrium. Furthermore, there is restoration of the proton path to an optimal level of functionality.

Cesium and rubidium are the two alkali metals with chemical and physical characteristics most similar to potassium. Potassium is the main internal cation of living cells. Potassium ion currents are central to the ionic physiology of normal viable healthy cells. Trans-membrane fluxes and cellular accumulation of cesium and rubidium ions are governed by similar cellular mechanisms as those which govern potassium movements; however, cesium and rubidium ions move at slower rates and accumulate to different degrees. Cesium and rubidium ions are effective for the control of potassium fluxes and linked hydrogen ions and other ionic fluxes making them essential ionic elements for Longevity Assurance therapy. (Cesium and rubidium ions are not available in the biosphere of western populations in sufficient quantities to promote therapeutic efficacy.)

### ADVANTAGES OF THE INVENTION

The present invention is unique in its approach to increasing longevity assurance through a targeted shifting of the ionic environment and pH regulation. This promotes an optimum or near optimum function of the biological system, restoring the ionic concentrations and function to obtain the optimum targeted values and ideal ranges for systemic pH, cellular pHe and cellular pHi and mitochondria pHe and pHi.

An advantage of the invention is that it treats and prevents the formation of an acidic/hypoxic biochemical environment, thus delaying and ameliorating the effects of age-related diseases, minimizing the accumulation of genetic damage within the cells and tissues of the body.

A further advantage of the present invention is that it can be cost effectively administered as a stand alone therapy or as an effective adjunct in conjunction with other therapies and nutrients to obtain expansion of mortality.

A further object of the present invention is to employ a fundamentally unique approach in modifying the systemic electro-physiological environment promoting optimum metabolic function.

A further advantage of the present invention is that it reduces or eliminates acidosis systemically lowering lifelong health costs.

A further object of the invention is to reduce acidotic hypoxia, stimulate the metabolism and balance changes in the cellular ionic environment, and to obtain the optimum conditions for cellular function reducing inflammatory response.

A further object of the present invention is to obtain localized and systemic genetic repair to maximize potential cell and mitochondria life span and delay cell senescence.

It is an object of the invention to increase metabolism, i.e., ATP production.

A further object of the invention is to provide a composition that reduces oxidative stress by preventing the formation of an acidic environment and reduces the production of free radicals.

Another object of the invention is to provide a cost effective intervention composition that restores critical fluids and electrolytes to reduce or prevent the lowering of the pH.

A further object of the invention is to promote extended cell division without mutation of the chromosomes, RNA and DNA and to extend replication cycles.

A further object of the invention is to obtain the optimum or near optimum pH, pHe and pHi values for glucose assimilation and ATP production providing DNA and RNA structure and function and repair activity.

A further object of the invention is to obtain a relatively constant pHe above 7.31, preferably ranging from 7.31 to 7.44, to optimize intracellular communication, disease resistance, and electro-physiologically correcting damage and mutations.

The present invention discloses compositions and methods for administering a pharmaceutical or nutraceutical composition to mammals, more specifically humans, for treating or preventing disorders associated with aberrant regulation of cellular homeostasis. The invention is based on prevention or treatment of disorders associated with a dysregulation of a generalized metabolic deficiency and cellular ionic homeostasis and disorders associated with pH reduction and aging. The composition may be administered to delay or ameliorate the effects of age-related diseases and to extend the lifespan of healthy, viable tissues and organs. The present invention provides a therapeutic composition containing alkaline ions to repair genetic damage occurring independently and randomly within trillions of cells.

The therapeutic composition minimizes the accumulation of genetic damage with the cells and repair.

### DETAILED DESCRIPTION OF THE INVENTION

“pH” refers to the negative logarithm of the Hydrogen ion activity: pH =−log (H+). Acidity and alkalinity are measured by pH. The parameter pHe is the pH on the exterior and pHi is the pH on the interior of the cell.

“Free radical” refers to an atom or molecule with an unpaired electron, that is, the electron is not paired with another electron in the formation of a chemical bond. Generally, this results in a molecule with a net magnetic movement, so it is paramagnetic. Free radicals are unstable, and they damage other chemicals, including DNA and RNA, etc.

Free radicals can be generated by ionizing radiation and a variety of chemical reactions within the cell. The main source of free radicals are redox reactions of oxidative energy metabolism considered by most biologists to be the primary cause of aging and disease onset. For example, donation of an electron to molecular oxygen can lead to generation of superoxide, oxygen free radicals, and hydroxyl radicals and peroxide that cause damage. These related oxygen toxins result in oxidative stress.

“Oxidative stress” refers to the biochemical insult to a cell resulting from damaging reactions within the cell involving oxygen. Abundant oxygen is clearly necessary to the energy metabolism of the cell, but the secondary reaction must be minimized and harmful chemicals detoxified.

“Effective amount” refers to an amount or quantity of an active substance that is sufficient to elicit the required or desired therapeutic response.

“Acid-forming reaction” refers to a reaction that is produced by any chemical reaction that affords a decreased ability to energize the biological system and leaves an acid residue, such as hydrogen ions (H+). The result is systemic and localized acidosis-induced hypoxia, a major cause of a wide variety of degenerative diseases and premature aging.

Virtually all electro-physiological processes are controlled by pH. In the normal course of biochemical events, there will be some degree of free radical generation. Free radical generation will be enhanced by any condition in which dys-electron donation occurs. Such biochemical errors are substantially increased in an acidic hypoxic biological environment (reduced pH) in which the supply of reductant to the electron transport chain exceeds electron acceptor capacity, or in a condition in which dysregulation of metabolism occurs. This occurs with any electro-physical condition that alters the biochemistry of regulation of metabolism, and most certainly occurs if H+ or other ion gradients linked to energy metabolism are in an incorrect electro-physical state. Thus, the efficient operation of these electrochemical and electro-physical systems depends primarily on optimum pH ranges, correct systemic pH, pHe and pHi.

Since some level of free radical generation is unavoidable, it is essential to have a method and composition employing electro-physical mechanisms that detoxify free radicals and their propagation environment and other harmful reactive chemical species. Protective mechanisms to inhibit the aging process involve antioxidant chemicals such as ascorbic acid and vitamin E, and detoxifying enzymes such as S.O.D.

The prior art is generally unaware of the underlying electro-physiological mechanism of aging and repair. Note, some of the genetic damage caused by radiation is identical to the free radical hypothesis of aging.

Cells have the electro-physical ability to locate and repair genetic damage acquired over the course of a lifetime, provided that the proper pH, ions, minerals, hydration, enzymes and sufficient nutrients are available.

The prior art is generally unaware of the implications and the critical role of pH, ionic physiology and has primarily concentrated on drugs employing technology that has severe limitations. For example, delivery to the site where the genetic damage occurs (inside the nucleus of the cell where the DNA is located) still remains a technical challenge for this approach. To be effective, the protective compounds in the drug must either survive the digestive process of the stomach or be converted by digestion to an active form of the drug that actually provides the therapeutic effect. The drug must have properties that encourage it to be absorbed into the bloodstream and be delivered to the cells. Upon arriving at a cell, the drug must then be capable of passing through the outer membrane of the cell and the inner membrane. The size, shape and electrical charge of these drugs often does not permit passage though these membranes without altering one or more of the physical characteristics that influence its therapeutic or protective effectiveness, and these limitations often cause negative side effects to the cell and organs.

The present invention provides a composition containing salts of cesium and rubidium, or a combination of both, to repair genetic damage occurring independently and randomly within trillions of cells. The therapeutic composition minimizes the accumulation of genetic damage with the cells of the body.

The method and composition is based on prevention and/or treatment of disorders associated with senescence, including Alzheimer's disease and cells and tissues associated with inflammation or the immune response, such as macrophages, bone marrow, lung (asthma), small intestine (Crohn's disease) and skin (erhthema nodosum). Examples of cell lines promoted by the therapy include, but are not limited to, fibroblasts, coronary endothelial cells, neuronal precursors, cardiac smooth muscle cells and promonocyte cell lines. Further examples of age-related diseases include, but are not limited to, cardiovascular disease, diabetes mellitus, neurological disorders, bone spurs, gallstones, indigestion, high blood pressure, arthritis, muscle cramps, gout, cholesterol, insomnia, Fibromyalgia, chronic fatigue syndrome, headaches, osteoporosis, Pick's disease, myotonic dystrophy, Huntington's disease, Parkinson's disease, multiple sclerosis, adult onset leukodystrophy, arteriolosclerosis, autoimmune diseases, etc.

The present invention discloses compositions and methods for prevention and or treatment of diseases and disorders associated with dysregulation of cellular homeostasis, cell cycle regulation, and the regulation of the balance between cell proliferation and apoptosis.

The composition may be administered with initial loading dosages and then administered as a maintenance or preventive dosages. The method employs a composition to electro-physically restore electro-physiological homeostasis, cell function. This corrects the metabolic deficiency associated with aging and thereby optimizes the cell growth environment and electro-chemical replication environment.

The composition of the present invention includes salts containing cesium and/or rubidium ions to neutralize the acidic biological environment. The method and composition's activity is electrochemical and electro-physical for obtaining therapeutic efficacy in gene stabilization, expression and repair. It restores the suboptimal pH that propagates free radicals, which are the cause of many undesirable effects of aging. Free radicals are unstable; they assault and damage cells, tissues and organ, thereby causing aging and contributing to a further reduction of the pH.

The pHe and pHi of the general population is sub-optimal. The associated, suboptimum pHe (acidosis) is a condition commonly associated with a wide variety of physiological and pathological situations. The method and composition reduces or eliminates the free radicals that occur in a reduced pH environment.

Rubidium ions provide the ideal intercellular pHi and the pHe for the mitochondria. The ions are selectively transported across cellular membranes by magnetic differential, thus tailoring the actives ions (medicine) to the patient's systemic pH, cellular pHe, pHi electro-biological age, physical condition, disease resistance and bio-chemistry. pHe ranges from 7.35 to 7.44. The near optimum pH of arterial blood is 7.40; venous blood is 7.35. Normal urine pH is about 6.00. Healthy saliva pH ranges between 7.00 and 7.50, more preferably 7.35 to 7.45.

Cesium and rubidium ions increase cellular metabolism. When the ions accumulate (due to electrogenic uptake or cation exchange mechanisms) in intracellular fluids, pHi is increased in cells, organs and tissues. The cells accumulated acidic substances are eliminated. In other words, pHe acidosis increases acute inflammatory response and induces human neutrophil activation. Such a response is instrumental in gene repair, expression, regulation and stabilization, and extends cell lifespan and cell replication cycles to obtain Longevity Assurance.

The systemic, local and/or cellular ionic physiology is altered by the electro-physical action of cesium and/or rubidium ions and the supporting electrolytes. The alteration of the ionic physiology reduces or delays and ameliorates the effects of a wide variety of age-related metabolic deficient diseases.

The present invention discloses compositions and methods that promote systemic pH elevation and promote a reduction of H+ (ions). This provides systemic and cellular resistance to disease invasion, producing ionic changes in the systemic pH, pHe and pHi, and changing the ionic chemistry of the cell as well as mitochondrial pHe and pHi. The composition eliminates and prevents the accumulation of acidic toxins and the acidic and or hypoxic energy metabolism necessary for disease progression and onset.

Clinical observation and diagnostic testing are used for adjustment of the dosages, in order to fall within optimal targeted systemic pH, pHe and pHi ranges. For example, saliva pH should range from 7.00 to 7.50, preferably targeting 7.30 to 7.50, during the therapy and only very briefly exceed 7.70.

By restoring the ionic function, the systemic pH, pHe and pHi are optimized and stabilized. The present composition promotes effects including those secondary to the inhibition of the large trans-membrane potassium movements resulting from acidosis energy metabolism. A sufficient dosage or dosages of the composition is administered to patients to obtain a targeted increase in the pH ranges, correction of excessive sodium accumulation, cell and organ hydration, modification of membrane electrical potential and improvement of capacity of the ion exchange mechanisms. Such dosages also restores optimal functioning of the proton path.

Dehydration is a primary factor in the formation of acidic toxins and a reduced pH. Free radicals propagate in an acidic environment, which increases oxidative stress, and the acidic pH stimulates distortion of cellular shape.

The invention discloses a composition and method that enhances the ability of healthy viable cells and normal tissue to tolerate and resist decreased pHe to the transport of H+ (ions) across its membrane and provides an electro-physical barrier to disease resistance and invasion.

The composition has a high efficacy-to-toxicity ratio, enabling a large portion of the treatment to be on an outpatient basis, resulting in substantial cost savings by reserving costly in-patient testing and therapies for patients with more advanced conditions.

Method for Ionic pH Therapies

The active ingredients of the composition are composed of salts of the alkali metals cesium and rubidium. The anionic moieties of the salts can be any nontoxic element or compound that does not substantially prevent biological availability of the cesium and/or rubidium ions. The cesium and/or rubidium salt compounds of the present invention may be employed either alone or as an adjunct with conventional therapies and can be utilized in a variety of ratios and concentrations to obtain Longevity Assurance. An initial loading dosages may be required to stabilize the reduced pH and rapidly elevating (increase) the pH; the composition is then administered as a maintenance or prophylaxis dosage.

Other ingredients are chosen to complement or potentiate the effect of the cesium and/or rubidium alkali salts. These other ingredients may alter the ionic metabolism of the pH environment, provide nutrient stimulation for normal viable healthy cells, or they may increase the tolerance or reduce stress of the patient.

For oral administration, the aqueous medium used in the manufacture and/or suspension of the composition may be processed to enhance the hydration. For example, the aqueous medium may be processed by electro-chemical activation (E.C.A.) obtaining a negative redox potential, promoting the neutralization of electrophilic toxins, and maintaining a redox potential in which the composition's ions, minerals and/or nutrients are processed. Typically, the surface tension ranges from 30 to 73 dynes per cm², preferably 40 to 60 dynes per cm², and the ORP ranges from −10 m.v. to −400 m.v., preferably −50 m.v. to −200 m.v. after dissociation. Typically, the pH of the composition ranges from 7.00 to 9.90, preferably 8.00 to 9.10, more preferably 9.30 to 9.50.

Where the composition is formulated as an I.V. drip, the ORP typically ranges from −300 m.v. to −380 m.v., with −320 m.v. to −360 m.v. being preferable, with a pH ranging from 7.20 to 7.50.

Therapeutic Methodology for Expansion of Mortality.

The efficacy of the composition during therapy is monitored by clinical observations and diagnostic particular to ionic therapy, including the monitoring of the saliva pH, pHe, pHi and systemic pH. Observations and therapies related to the physiological stresses of Ionic pH therapy are included to prevent excessive therapy-related stress.

Use in Prevention of Age-Related Diseases and Expansion of Mortality.

The composition reduces and eliminates the development of acidosis, (a reduced pH). Other parameters of ionic metabolism can be measured at a cellular level, such as cytoplasmic pH.

Toxicity is not a significant factor at the lower nutraceutical or bioceutical or maintenance dosages. At such dosage levels, there are no significant stresses associated with ionic therapy for expansion of mortality, age-onset prevention, or delaying age-related diseases.

### BEST MODE FOR CARRYING OUT THE INVENTION

The preferred embodiment provides compositions and methods for reducing or eliminating acidic hypoxias activity and restoring metabolic function for aging intervention and obtaining expansion of mortality.

The method and composition increases the systemic pH and lowers the acidity levels of the pHe and pHi in the biological environment. When the acidity of the cellular environment is elevated to a more electro-physiologically optimum pHe level above 7.31, the patient's metabolic and immune systems (including antibodies, macrophage cells, etc.) function more efficiently. The Cesium ions generally shift the systemic pH more rapidly than the Rubidium ions. The rubidium ions increase ATP production, stimulating metabolism. Rubidium ions shift the cellular pHi more effectively.

Such a response is instrumental in the suppression of the effects of age-related diseases. If a patient's immune system is suppressed by a systemic or localized reduced pH, (acidic hypoxia biological environment), the therapy described herein will stimulate the immune response by a targeted increase of the systemic pH, pHe and pHi to optimum range during the therapy. This will accordingly stimulate the patient's immune response and function to resist a wide variety of infections and diseases. Obtaining the targeted physiologically optimum pHe and pHi ranges optimizes intracellular communication.

Principal Active Ingredients and Method of Manufacture

This invention utilizes salts containing ions of cesium and/or rubidium, in a wide variety of ratios or combinations, in its manufacture, including, but not limited to, cesium chloride and rubidium chloride. The compositions and methods employ a synergistic alkaline salt or a solution formed by the following chemical composition: “MA”, where “MA” substantially dissociates in water solution to form “M+” and “A−”. “M” is the alkali metal moiety, which may be cesium and/or rubidium. “A” is the anionic moiety, which may be any compatible nontoxic inorganic species, such as, but not limited to chloride, sulfate, carbonate or phosphate etc.; or it may be any nontoxic any nontoxic organic species such as lactate, citrate or acetate, etc. The composition typically includes 100 ppm to 20,000 ppm of a cesium or rubidium ion source or combination of the two.

In the event that it may be desired to combine the alkali metal moiety with an anionic moiety with which it is not readily commercially available, this can also be accomplished. For example, the hydroxide of the alkali metal can be combined with the acid form of the desired anion, thus: “MOH+HA=MA+H₂O”. In the case of acids that can dissociate more than one hydrogen ion, the final product may be partially protonated, for example, “MHCO₂”, the bicarbonate, or “M₂CO₂”, the carbonate. The final product can be formulated by controlling the stoichiometry of the reaction, or by any known manufacturing process to obtain the required final pH, ORP (Oxidation Reduction Potential) and required ionic ratios and concentrations.

To decrease non-limiting systemic acidity and elevate systemic pH, pHe and pHi, carbonate or an organic species that can be readily metabolized are preferred. For example, citric acid can be used to neutralize a solution of cesium hydroxide until a pH near neutrality is obtained, or precise amounts of cesium hydroxide can be mixed with predetermined amounts of citric acid so that on dissolution, a predetermined physiologic pH will be obtained.

For oral administration the palatability will influence choice of anion(s) and the flavoring agent or other agents employed.

The proportion or ratios of cesium to rubidium salts employed will be governed by the requirements for the composition's efficacy and dosages and the physiological stress on the patient.

The pharmaceutically acceptable salt or salts of the present invention can be synthesized from the parent compound, which contains a basic or acidic moiety by conventional chemical methods. Generally, such salts can be prepared by reacting with the free acid or base forms of these compounds with a predetermined amount of the appropriate base or acid in water or in an organic solvent, or in a mixture of the two.

Any combination of cesium and/or rubidium salts which dissociate and ionize may be employed in the composition of the present invention, including, but not limited to: Arginate, Ascorbate, Aspartate, Caprylate, Chloride, Cysteinate, Citrate, Fumarate, Humic, Fulvate, Methionine, Glutamate, Gluconate, Glycinate, Aspartate, Lysinate, Succinate, Carbonate, Lactate, Malate, Malic, Tartrate, Sulfate, Phosphate, Nitrate, Flouride, Bromide, Iodide, Orotate, Asporotate, Bisulfonate, Lysinate, Fulvic, Succate, Carnate, Trisulfate, Lactobionate, Benzenesulfonate, Laurate, Benzoate, Bicarbonate, Benzoic, Caseinate, Bisufate, Mandelate, Bitartrate, Mesylate, Borate, Methylbromide, Methylnitrate, Calcium Edetate, Methylsulfate, Camsylate, Mucate, Napsylate, Clavulanate, N-Methylglucamine, Ammonium Salt, Dihydrochloride, Oleate, Edetate, Oxalate, Edisylate, Pamoate (Embonate), Esolate, Palmitate, Esylate, Pantothenate, Phosphate/Diphosphate, Gluceptate, Polygalacturonate, Salicylate, Stearate, Glycollylarsanilate, Hexylresorcinate, Subacetate, Hydrabamine, Hydrobromide, Tannate, Hydrochloride, Hydroxynaphthoate, Teoclate, Tosylate, Isothionate, Triethiodide, Panoate, Valerate, Acetate, Maleate, Malonate, Sulfate, and mixtures thereof.

Alternately or additionally, other salts might be used, including various organic or metallic salts, if they meet the following requirements: (1) they must be pharmaceutically acceptable and have a low level of toxicity; (2) they must have sufficiently high levels of solubility in water or a saline solution; (3) they must allow the salt to be infused into the patient in a stable and slightly alkaline solution having a pH range from 7.20 to 7.50, in water or a saline solution; and, (4) they must have sufficiently high levels of cationic (alkaline) dissociation to allow the remaining negatively charged molecules to effectively reduce the acidity of cells and tissues. Lists of suitable salts are found in Remington's Pharmaceutical Sciences, 17^{th} ed., Mack Publishing Company, Easton, Pa. 1985, p. 1418, the disclosure of which is hereby incorporated by reference.

Secondary Ingredients

These ingredients are chosen to complement or potentiate the action of the active cesium and or rubidium ions. Some examples of potentiating ingredients are given to instruct the physician in the principals of their selection and are not intended to exclude other ingredients not mentioned. Potentiation of cesium and/or rubidium ionic action can be accomplished by inclusion of ingredients that enhance ionic pH physiology. Examples are compounds that stimulate calcium accumulation, such as calcium supplements and magnesium, preferably in an equal ratio, vitamin D3, selenium salts, calcitonin, calcium ionophores, etc. Other compounds include humic and fulvic acid and glycol-sugars.

Another class of supportive ingredients which potentiate the activity of the cesium and rubidium ion and support the immune system and healthy viable cells, especially those which may be deficient as a secondary consequence of age-related effects, such as potassium, magnesium, manganese, zinc, vitamin B2, B6 (pyridoxine) and B12 (cyanocobalamin). Ingredients that complement the salts of cesium and/or rubidium therapy are those that acts by unrelated means but which may be useful. Additional classes of ingredients which complement the ionic actions of rubidium and/or cesium salts therapy are those which provide a balance of the primary ions needed to carry on metabolic processes. These electrolytes are balanced to match the concentration ratios of the electrolytes that require supplementation in a particular patient. Potassium, the primary ion, with the body's other major electrolytes, sodium, calcium, magnesium, chloride, bicarbonate, phosphate, and sulfates, are added to the formulation in proportion to the potassium. Manganese may be added for enzyme activation.

Where a potassium salt and a magnesium salt are included in a composition of the present invention, the weight ratio of potassium salt to magnesium salt is typically 1:1 to 2:1. The ratio is typically not greater than 3:1.

Mineral supplements including trace minerals and ions are also used to obtain and maintain the desired pH range of bodily fluids and optimize cellular structure, function and metabolism, etc.

In a preferred (non-limiting) embodiment of the present invention, may include more than 50, and more preferably, more than 70 trace minerals. These nutrients act to maintain optimal health while consuming a carefully designed, and perhaps minimal, food plan. These trace minerals are also a great benefit to those persons who have actual (or body perceived) mineral deficiencies which, unsatisfied, can increase cravings for foods. The inclusion of these trace minerals decreases the need to identify the precise deficiency of minerals, and supplies the necessary supplementation easily.

Enzyme therapy may be included if appropriate. Calorie restriction may be included if appropriate, typically with clinical observation.

Physical Form

If the composition includes salt or salts containing ions of cesium and/or rubidium for oral administration, it may be manufactured by conventional methods with supporting electrolytes. The composition may be orally administered without previous dissolution or prepared as an aqueous solution suitable for ingestion or slow IV injection using a carrier liquid. As an example, solutions for injection may be prepared with a chemical composition that renders the solutions pH balanced and acceptable for injection, in a sterile, buffered saline solution isotonic to blood.

The composition may be prepared as an oily injection suspension. Suitable lipophilic solvents or vehicles include, but are not limited to, fatty oils such as sesame oil, or synthetic fatty esters, such as ethyl oleate or triglycerides, or liposomes, etc.

Method of Use/Modes of Administration

The composition utilized in this invention may be administered by any acceptable route including, but not limited to, oral, periodic injections, intravenous infusion, topical, and sublingual. Oral or IV routes are preferred.

The alkaline salt or salts described herein may be administered orally in a fluid medium, or in a powdered tablets or capsules, etc.

Preparations for oral administration include push-fit capsules made of gelatin, as well as soft, sealed capsules made of gelatin and a coating, such as glycerol or sorbitol. Push fit capsules can contain the composition with electrolytes mixed with a filler or binders, such as cellulose lactose or starches, etc., or with nutrient additives. For oral administration, the capsules may be administered simultaneously with sufficient water with a suitable complex carbohydrate.

The composition may take the form of gels, oils, bandages/dressings, topical lotions, douche solutions, suppositories, colon irrigation solutions, drop dispersions, encapsulation in liposomes (including time-release lyposomes), micro-particles, enteric coatings, microcapsules, transdermal patches, etc.

In general, the prescribed dosage(s) required for Ionic pH therapeutic efficacy for and Expansion of Mortality will be dependent on such factors as the patient's initial or starting saliva pH, urine pH, exact venous pH, weight, age, diet, nutritional background, gender, physical symptoms and general health (condition), duration and frequency of administration, chosen route of administration, reaction sensitivities, drug combination(s), and tolerance, and clinical condition and severity of the diseases.

Compositions suitable for use in the invention include compositions wherein the active ions and electrolyte ingredients are contained in an effective amount to achieve the intended purpose. The determination of an effective dose is well within the capability of those skilled in the art.

The composition may be administered every 8 to 24 hours.

The composition and dosage(s) are adjusted (increased or decreased) as therapy progresses. For example, a patient suffering from a generalized metabolic deficiency, i.e., reduced pH (acidosis) may be treated to obtain Longevity Assurance with salts containing cesium ions and/or rubidium ions or in any combination to delay or ameliorate the age-related effects and postpone or prevent the pH from falling to suboptimal levels.

Dietary Intervention

In the clinical environment, the method and composition are most effective if the patient's diet is nutritionally stimulating and does not contribute to acidotic stress. As an example, dietary foods and beverages with a pH below 2.50 should be completely eliminated; Low glycemic index foods are preferred during therapy. Oral fluid uptake should be derived primarily from water with a pH ranging from 7.0 to 9.5, preferably gradually increasing the pH of the oral fluids from 7.00 to 9.40.

Any foods with pH below 3 and beverages and foods whose low pH results from mineral acids such as phosphoric acid should be minimized. A neutral or slightly alkaline diet should be administered during the therapy.

Efficacy

The patient's saliva, urine and blood pH should be monitored during the clinical treatment process and the dosage appropriately adjusted. The goal of dosage adjustment is to partially or wholly restore and maintain the optimum physiologic pHe ranges between 7.31 to 7.45 and elevating the pHi above 6.40, preferably between 6.60 to 6.80.

Generally, the saliva pH reflects the ionic concentration and composition of the plasma; the saliva pH will gradually increase the pHe it two to three weeks. Note, clinical dosages elevates the saliva pH in 20 to 40 hours.

A saliva pH between 7.10 to 7.50 indicates generally good health. The saliva pH and pHe and pHi, and other indicating features of ionic pH therapy such as sodium, potassium, magnesium and calcium levels and metabolites such as lactate can be monitored.

A lack of adequate pH increase or therapeutic response of the saliva pH or other indicators suggests dehydration and/or insufficient dosage or lack of sufficient dietary intervention or an acute diseased state.

Stress

Excessive doses of salts containing ions of rubidium and/or cesium can cause physiological stress; examples are: the mild diuretic effects, potassium depletion, low blood pressure or excessive alkalization. In extreme cases where maximal efficacy is required during clinical treatment, the upper dosage limit is set by the stress symptoms. The maximum dosage must be below the point at which perturbation of electrolyte balance causes damage.

The pH measurements previously stated for efficacy will provide information useful for the physician in assessment of physiological stress. The patient should be well hydrated before administering the composition. Dehydration should be monitored and promptly corrected. Blood pH should not rise above 7.45; the targeted optimum saliva pH should range from 7.3 to 7.50 and only very briefly rise above 7.60.

The urine pH should not rise above 7.20 or drop below 5.00 and only very briefly fall below 4.50. Additional symptoms of excessive dosages and excessive pH elevation is a slight numbness around the mouth, and further progresses to muscle aches. These symptoms indicate the need for a temporary reduction in the dosage to avoid excessive alkalosis-induced stress, with increased dosage of potassium and magnesium with electrolytes, and an increased fluid uptake until the symptoms are reduced to a manageable therapeutic level.

Doses should not exceed those that cause only slight sensations of numbness on the upper lip and around the mouth during therapy. Excessive sensations of numbness indicate incipient effects on nerve tissue's ionic status. Blood potassium should not fall below tolerable levels. Blood pressure and serum creatine levels should be monitored.

Exemplary Compositions

Nutraceutical Beverage

In one aspect of the present invention, the composition is in the form of a nutraceutical beverage. The nutraceutical beverage provides, for example, enhanced hydration. The beverage contains water, typically electrolyzed water, and a cesium salt, and or rubidium salt or a combination of the two.

Other supporting ingredients that may be included in the nutraceutical beverage include, without limitation, potassium, magnesium with other electrolytes, vitamins and other support nutrients, potassium (preferably as phosphate, gluconate and/or acetate), calcium, magnesium citrate, manganese (citrate or orotate), iodine, selenium, vanadium (vanadyl sulfate), zinc (gluconate/asporotate), Vitamin D3 (cholecalciferol), Vitamin A, Vitamin C (buffered L-ascorbic acid), malic acid, Coenzyme Q 10 (ubiquinone), B3 (methyl nicotinate), B2, B6 (pyridoxine), B12 (cyanocobalmin), and Superoxide dismutase.

In a specific case, the composition includes the following ingredients: Amounts per 10 to 30 ounces bottled in electrolyzed an aqueous solution containing cesium and/or rubidium salts with potassium, magnesium with other electrolytes, vitamins and other support nutrients, such as, cesium carbonate and/or rubidium carbonate, or any combination or ratios thereof, ranging from 10 mg per 24 hours to 150 mg per 24 hours, preferably 25 to 150 mg per 24 hours; potassium (preferably as phosphate, gluconate and/or acetate) 100-500 mg; calcium 500 to 2,500 mg; magnesium citrate 200-1500 mg; manganese (citrate or orotate) 1 to 20 mg; iodine 10-40 mcg.; selenium (Selenomethionine) 5-20 mcg; vanadium (vanadyl sulfate) 1-5 mg; zinc (gluconate/asporotate) 3-20 mg; Vitamin D3 (cholecalciferol) 1,000 to 4,000 IU; Vitamin A 1,000 to 3,000 IU; Vitamin C (buffered L-ascorbic acid) 500 to 1,500 mg; Coenzyme Q 10 (ubiquinone) 10-30 mg; B3 (methyl nicotinate) 20-30 mg; B6 (pyridoxine) 25-100 mg; B12 (cyanocobalamin) 10-100 mcg. Superoxide dismutase 10 to 1,000 mg, preferably 50 to 100 mg.

In another specific case, the composition includes the following ingredients in water, preferably in an electrolyzed aqueous solution: cesium salt (50-150 mg); potassium salt (50-300 mg); and, magnesium salt (25-150 mg).

In another specific case, the composition includes the following ingredients in water, preferably in an electrolyzed aqueous solution: cesium chloride (50-150 mg); potassium citrate (50-300 mg); and magnesium citrate or ascorbate (15-150 mg).

In another specific case, the composition includes the following ingredients in water, preferably in an electrolyzed solution: cesium chloride (100 mg); potassium citrate (100-150 mg); and magnesium citrate or ascorbate (50-75 mg).

In a system aspect of the nutraceutical beverage, a container containing the beverage is provided. The container includes instructions, either attached to the container or provided with it, regarding the proper way to ingest the beverage for optimal effect. Preferably, the container includes instructions related to the appropriate amount of beverage to ingest to achieve optimal effect (e.g., between 10 mg to 150 mg of cesium and/or rubidium salt per 24 hour period).

Capsules or Tablets

In another aspect of the present invention, the composition is in the form of a capsule or tablet. The tablets, when taken over a period of time, serve to alkalinize a subject's body, which increases athletic performance. Indicia of athletic performance increase include, without limitation, decreased soreness after activity, decreased recovery time, and increased stamina. The tablets include a cesium salt, a rubidium salt or a combination of the two.

Typically, doses of the cesium or rubidium salts range from 10 mg to 100 mg per 24 hours, up to a total dose of about 250 mg per 24 hours. Other ingredients that may be included in the tablets include, without limitation, cesium citrate, rubidium citrate, potassium (preferably as phosphate, gluconate and acetate), calcium (carbonate), magnesium citrate, manganese, iodine, selenium (Selenomethionine), vanadium (vanadyl sulfate), zinc gluconate, Vitamin D3, Vitamin A, Vitamin C (buffered L-ascorbic acid), malic acid, Co-enzyme Q 10 (ubiquinone), B3 (methyl nicotinate), B6 (pyridoxine), B12 (cyanocobalamin), and superoxide dismutase.

In a specific case, the composition includes the following ingredients: cesium and/or rubidium salts administered in tablet or capsule ranging from 20 mg to over 100 mg, generally 25 mg to 100 mg per 24 hours is orally ingested with alkaline water combined with a suitable complex (slow-burning) carbohydrate. The nutrient support is preferably to be orally administered as one or more tablets or capsules per 24 hrs, depending on dose or dosages of tablet or capsule as in the following, maximal ranges (the dose is oftentimes substantially lower): iodine 150-400 mcg.; selenium (Selenomethionine) 50-200 mcg; vanadium (vanadyl sulfate) 2-10 mg; zinc gluconate 50-200 mg; Vitamin D3 2,000 (cholecalciferol) to 4,000 IU; Vitamin A 2,000 to 5,000 IU; Vitamin C (buffered L-ascorbic acid); 500 to 2,000 mg; malic acid 3-5 mg; Co-enzyme Q 10 (ubiquinone) 25-50 mg; B3 (methyl nicotinate) 5-20 mg; B6 (pyridoxine) 25-100 mg; B12 (cyanocobalamin) 20 to 50 mcg; superoxide dismutase 100 to 2,000 mg. Preferably 250 to 500 mg.

In another specific case, the composition includes the following ingredients (typically in capsule form): cesium salt (50-150 mg); potassium salt (50-300 mg); and, magnesium salt (25-150 mg).

In another specific case, the composition includes the following ingredients (typically in capsule form): cesium chloride (50-150 mg); potassium citrate (50-300 mg); and magnesium citrate or ascorbate (15-150 mg).

In another specific case, the composition includes the following ingredients (typically in capsule form): cesium chloride (100 mg); potassium citrate (100-150 mg); and magnesium citrate or ascorbate (50-75 mg).

In a system aspect of the capsules or tablets, the capsules or tablets are included in a container. The container includes instructions, either attached to the container or included with it, regarding the proper way to ingest the capsules or tablets for optimal effect. Preferably, the container includes instructions related to the appropriate dosage to ingest to achieve optimal effect (e.g., cesium or rubidium salts ingestion ranging from 10 mg to 100 mg per 24 hours, up to a total dose of about 250 mg per 24 hours).

Effervescent Tablets or Granules

In another aspect of the present invention, the composition is in the form of effervescent tablets or granules. The effervescent quality aids in the dissolution of the other ingredients of the tablets or granules.

Effervescent tablets or granules with effervescent effect, typically include at least one acid component and one gas-evolving component of alkali hydrogen carbonate, alkali carbonate, and/or alkaline-earth carbonate particles evolving gas under the action of acid, as well as of active salts of cesium and rubidium, with potassium, magnesium and other electrolytes, fragrances, colorants, plant extracts, vitamins, minerals and trace minerals admixed as needed, with at least one acid component combined with at least one of the following compounds: alkali carbonate, alkali hydrogen carbonate, alkaline-earth carbonate, alkaline-earth oxide, hydrocolloid, or mixtures of glycols, etc. Additional calcium carbonate can be added to this phase in order to obtain a higher calcium dose if required.

In a specific case, the effervescent composition includes the following ingredients: cesium salt (50-150 mg); potassium salt (50-300 mg); and, magnesium salt (25-150 mg).

In another specific case, the effervescent composition includes the following ingredients: cesium chloride (50-150 mg); potassium citrate (50-300 mg); and magnesium citrate or ascorbate (15-150 mg).

In another specific case, the effervescent composition includes the following ingredients: cesium chloride (100 mg); potassium citrate (100-150 mg); and magnesium citrate or ascorbate (50-75 mg).

Meal Substitute/Nutritional Bar

In another aspect of the present invention, the composition is in the form of a meal substitute or nutritional bar. Among other beneficial properties, the bar satiates hunger, which aids in weight loss and or control. The bars include a cesium salt, a rubidium salt or a combination of the two.

Carriers for the active salts in the bar may be of several different forms, including, without limitation, chocolate or carob, oats, wheat, peanut butter, semi-dried fruits, grains and combinations thereof; and a gelatin product where the carrier is in the form of gelatin and water containing electrolytes, various vitamins, minerals and trace minerals, coloring agents, flavors appropriate for weight maintenance or weight loss providing therapeutic benefit.

In a specific case, the meal substitute or nutritional bar includes the following ingredients: cesium salt (50-150 mg); potassium salt (50-300 mg); and, magnesium salt (25-150 mg).

In another specific case, the meal substitute or nutritional bar includes the following ingredients: cesium chloride (50-150 mg); potassium citrate (50-300 mg); and magnesium citrate or ascorbate (15-150 mg).

In another specific case, the meal substitute or nutritional bar includes the following ingredients: cesium chloride (100 mg); potassium citrate (100-150 mg); and magnesium citrate or ascorbate (50-75 mg).

### EXAMPLES

The examples below are provided to illustrate the subject invention and are not included for the purpose of limiting the scope of the invention.

### Example 1

### For Oral Administration of an Aqueous Nutraceutical Beverage

The dosage for salts of cesium and/or rubidium in therapy by oral aqueous administration. Amounts per 10 to 30 ounces bottled in an alkaline aqueous solution containing cesium and/or rubidium salts with potassium, magnesium with other electrolytes, vitamins and other support nutrients, such as, cesium carbonate and/or rubidium carbonate, or any combination or ratios thereof, ranging from 10 mg per 24 hours to 150 mg per 24 hours, preferably 2 to 50 mg per 24 hours; (the following doses are maximum doses and typically they are substantially lower) potassium (preferably as phosphate, gluconate and/or acetate) 100-500 mg; calcium 500 to 2,500 mg; magnesium citrate 200-1500 mg; manganese (citrate or orotate) 1 to 20 mg; iodine 10-40 mcg.; selenium (Selenomethionine) 5-20 mcg; vanadium (vanadyl sulfate) 1-5 mg; zinc (gluconate/asporotate) 3-20 mg; Vitamin D3 (cholecalciferol) 1,000 to 4,000 IU; Vitamin A 1,000 to 3,000 IU; Vitamin C (buffered L-ascorbic acid) 500 to 1,500 mg; malic acid 5-25 mg; Coenzyme Q 10 (ubiquinone) 10-30 mg; B3 (methyl nicotinate) 20-30 mg; B6 (pyridoxine) 25-100 mg; B112 (cyanocobalmin) 10-100 mcg. Superoxide dismutase 10 to 1,000 mg, preferably 50 to 100 mg.

The patient should be monitored for stress and efficacy as described herein, and dosages adjusted to obtain a pH elevation to targeted ranges or suitable response with minimal physiological stress. Insufficient response, either initially or after a period of favorable response, indicates that complementary or potentiating ingredients should be considered.

### Example 2

### Capsules or Tablets for Oral Administration

This composition is for maintenance dosages for Expansion of Mortality. The alkaline salts described herein may be administered orally, or in a tablet or capsule form, etc.

Oral preparations include push-fit capsules made of gelatin, as well as soft, sealed capsules made of gelatin and a coating, such as glycerol or sorbitol. Push fit capsules containing powdered salts of cesium and/or rubidium may contain electrolytes with fillers or binders, such as lactose or starches. Dosages of the salts containing cesium and/or rubidium ions for longevity assurance may range from 25 mg to 100 mg per 24 hours, preferably 50 mg to 200 mg per 24 hours, up to a total dose of about 250 mg per 24 hours.

As an example, but not limited to: cesium citrate 5 to 100 mg; rubidium citrate 25 mg; potassium (preferably as phosphate, gluconate and acetate) 50 to 100 mg; calcium (carbonate) 500 to 1,200 mg; magnesium citrate 50 to 500 mg; manganese 5 to 20 mg. The therapeutic dosage of the cesium and/or rubidium salts administered in tablet or capsule ranging from 20 to over 100 milligrams, generally 25 to 100 mg per 24 hours is orally ingested with alkaline water combined with a suitable complex (slow burning) carbohydrate. The nutrient support is preferably to be orally administered as one or more tablets or capsules per 24 hrs, depending on dose or dosages of tablet or capsule as in the following ranges (maximal doses listed, typically they are substantially lower): iodine 150-400 mcg.; selenium (Selenomethionine) 50-200 mcg; vanadium (vanadyl sulfate) 2-10 mg; zinc gluconate 50-200 mg; Vitamin D3 2,000 (cholecalciferol) to 4,000 IU; Vitamin A 2,000 to 5,000 IU; Vitamin C (buffered L-ascorbic acid); 500 to 2,000 mg; malic acid 3-5 mg; Co-enzyme Q 10 (ubiquinone) 25-50 mg; B3 (methyl nicotinate) 5-20 mg; B6 (pyrodoxine) 25-100 mg; B12 (cyanocobalamin) 20 to 50 mcg; superoxide dismutase 100 to 2,000 mg. Preferably 250 to 500 mg.

During the therapy the optimum targeted saliva pH ranges between 7.30 to 7.50, and should only very briefly rise up to 7.60. Urine pH should not rise above 7.00 to 7.20 or drop below 5.00 and only very briefly fall below 4.50.

### Example 3

### Effervescent Tablets or Granules with 2,500 Mg Calcium Exhibiting Dissolution Reaction when Introduced into Water Containing Salts with Ions of Cesium and/or Rubidium

A citric acid phase passivated with calcium carbonate may be used. The amount of calcium may additionally be contained in the carbonate phase. Either calcium carbonate granules or tablets or precipitated calcium carbonate may be utilized.

### Example 4

### Effervescent Tablets or Granules with Cesium and/or Rubidium Salts, Vitamins, Amino Acids, Manganese Potassium and Magnesium with Other Electrolytes

Effervescent tablets or granules with effervescent effect, consisting of at least one acid component and one gas-evolving component of alkali hydrogen carbonate, alkali carbonate, and/or alkaline-earth carbonate particles evolving gas under the action of acid, as well as salts of cesium and or rubidium, with electrolytes including potassium, magnesium, fragrances, colorants, plant extracts, vitamins, minerals and trace minerals admixed as needed, with at least one acid component combined with at least one of the following compounds: alkali carbonate, alkali hydrogen carbonate, alkaline-earth carbonate, alkaline-earth oxide, hydrocolloid, or mixtures of glycols, etc.

Additional calcium carbonate can be added to this phase in order to obtain a higher calcium dose if required.

With an additional amount of alkali carbonates or alkali hydrogen carbonates, one can also set the desired pH value. This basic mixture can be provided with suitable sweeteners if required, such as truting dulcem, as well as with fragrances, flavors and colorants. When needed, fillers which in their grain size distribution match the basic granules can be introduced.

The tablets or granules are dropped in to the bottom when introduced into clear water (not sodas), and effervesce, whereupon the soluble salts of cesium and rubidium and the electrolytes are dissolved.

### Example 5

### Oral Capsule or Tablets Containing Powdered Salts of Cesium and/or Rubidium

This formulation is intended for use by patients as a maintenance dose for lifelong aging-intervention therapy. The dosages are administered as one or more tablets or capsules per 24 hours taken with a meal. Dosage generally ranges from 20 to 100 mg, as an example, cesium citrate 25 mg; rubidium citrate 25 mg; potassium (preferably as citrate, gluconate and/or acetate) with other nutrients. As an example, foods and beverages with pH below 2.5 should be completely eliminated. Oral fluid uptake should be derived primarily from electrolytically alkaline processed water with a pH ranging from 7.5 to 9.3, preferably gradually increasing from 7.50 to 9.30.

### Example 6

Meal Substitute or Food Bars Containing salts of Cesium and/or Rubidium Ions. The compositions include food bars as a nutritional substitute or as a nutritious addition where the carrier is in the form of chocolate or carob, oats, wheat, peanut butter, semi-dried fruits, grains and combinations thereof; and a gelatin product where the carrier is in the form of gelatin and water containing electrolytes, various vitamins, minerals and trace minerals, coloring agents, flavors appropriate for weight maintenance or weight loss providing therapeutic benefit.

Cesium and rubidium ions used in the present are separate and distinct from man-made isotopes of cesium and rubidium.

1. A composition for increasing longevity of a mammal, wherein the composition comprises: a cesium or rubidium salt; ECA processed water; and, a potassium salt.

2. The composition according to claim 1, wherein the surface tension of the ECA processed water ranges from 30 to 73 dynes per cm².

3. The composition according to claim 1, wherein the pH of the composition ranges from 7.00 to 9.40.

4. The composition according to claim 1, wherein the ORP of the composition ranges from −10 m.v. to −400 m.v.

5. The composition according to claim 1, wherein the composition further comprises citrate.

6. A nutraceutical beverage composition, wherein the composition comprises: a cesium or rubidium salt; water; and, electrolytes.

7. The composition according to claim 6, wherein the composition further comprises superoxide dismutase.

8. The composition according to claim 6, wherein the composition further comprises vitamins B3, B6 and B12.

9. The composition according to claim 6, wherein the composition further comprises selenium, vanadium and zinc.

10. An effervescent composition, wherein the composition comprises: an alkali hydrogen carbonate, alkali carbonate, or alkaline-earth carbonate particles evolving gas under the action of acid; active salts of cesium or rubidium; potassium; and, magnesium.

11. The composition according to claim 10, wherein the composition further comprises fragrances or colorants.

12. The composition according to claim 10, wherein the composition further comprises plant extracts.

13. The compositions according to claim 10, wherein the composition further comprises vitamins and trace minerals.

14. A nutritional bar composition, wherein the composition comprises: a cesium salt or a rubidium salt; and, a carrier selected from chocolate, carob, oats, wheat, peanut butter, semi-dried fruits, grains and combinations thereof.

15. The composition according to claim 14, wherein the composition further comprises electrolytes and trace minerals.

16. The composition according to claim 14, wherein the composition further comprises coloring agents.
